(19) United States
(12) Patent Application Publication
(10) Pub. No.: US 2005/0095331 A1
(43) Pub. Date: May 5, 2005

(54) Process for obtaining bio-functional fractions from biomass

(76) Inventor: Douglas Van Thorre, Minneapolis, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/605,812

(22) Filed: Oct. 29, 2003

(51) Int. Cl. C12P 19/14; A23P 1/00; B02B 3/12

(52) U.S. Cl. 426/478; 241/8

(57) ABSTRACT

The present invention includes a method for separating corn germ from the remaining corn matrix. The method includes providing a corn kernel; dry milling the kernel and treating the milled kernel in an isotonic solution to separate the germ from the corn matrix.

One embodiment of the present invention includes a method for separating germ from a corn kernel. The method includes wetting a corn kernel prior to transferring the kernel to a mill and milling the kernel. The milled corn kernel is then dispersed in a vessel containing an aqueous isotonic solution having a density and volume effective for floating the corn germ and sinking the remaining corn matrix. The floating corn germ is skimmed at or near the top of the solution level in the vessel. The remaining corn material is extracted at or near the bottom of the vessel. In one embodiment, one or more of the milling and isotonic separation is performed as a continuous process. In one embodiment, the isotonic solution is prepared using a material that is usable in other operations of the process. For instance, fermentable sugar is usable to prepare the isotonic solution. Once the fermentable sugar is used as a separating isotonic solution, the spect solution is usable in other operations of the overall process. For instance, the spent isotonic solution that includes fermentable sugar is also usable as feedstock to a fermentor. This embodiment of a corn germ separation process is then a continuous, closed loop, zero discharge process.

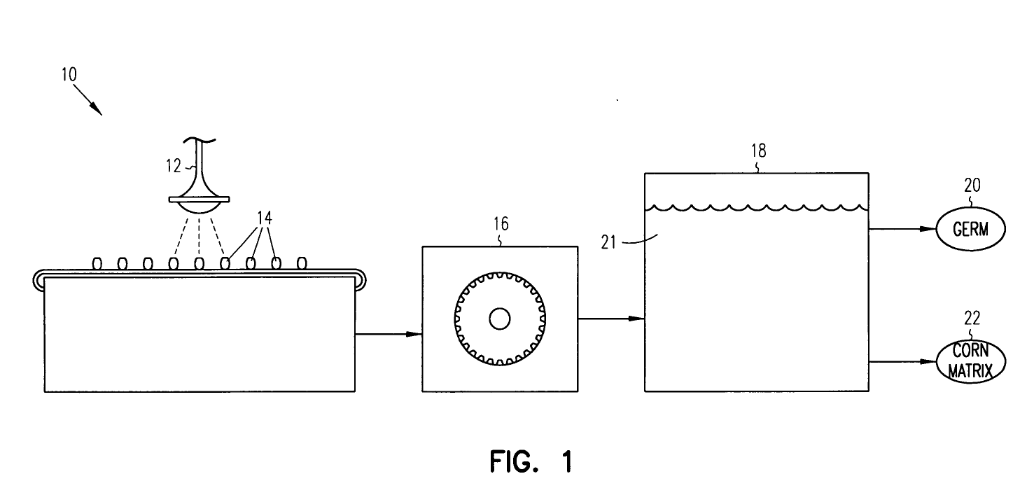

One system embodiment of the present invention, illustrated schematically at 10 in FIG. 1 includes a wetting device 12 for wetting kernels, one kernel of which is at 14, prior to dry milling the kernels; a mill 16 for performing dry milling and a vessel 18 for containing the isotonic solution 21 and for separating corn germ 20 from the remaining corn matrix 22. One suitable kernel wetting device includes a nozzle capable of delivering water as a spray to kernels that are on a moving bed or are in a hopper. While a kernel wetting device is described for one embodiment, it is understood that other system and method embodiments do not include a wetting device. The kernel milling is performed using mills and other equipment typically used in a dry milling process. The corn kernels are wetted prior to milling in order to transport the kernels to and through a mill and to reduce the amount of corn particle dust generated and to reduce or eliminate requirements for dust collection equipment.

The germ separation is performed in a dry milling process of the invention. Dry milling does not damage the pericarp and starch. Consequently, these materials are capable of being subsequently processed into value added products.

The corn germ contains approximately 85% of the oil found in the virgin corn kernel. Separating the corn germ improves the extraction and recovery of the oil from the germ and the processing of protein in the germ. The corn oil is not present in subsequent process steps to interfere or complicate the further processing of the corn kernel complex which is generally made up of the pericarp and starch.

The process embodiments described herein combine the best of the wet mill and dry mill processing to have a process that separates the corn germ cleanly and does not damage the pericarp and starch. As a consequence, the pericarp and starch are optionally processed into value added products. The process embodiments of the invention use dry corn germ separators, milling devices, which have a long history of performing relatively well in separating the corn germ from the pericarp/starch complex. The difficult part has been the separation of the corn germ from the remaining material after the degerming process, in an economical manner. This is accomplished by embodiments of the invention described herein.

Once the germ is separated from the pericarp/starch complex, the pericarp/starch complex is subjected to size reduction and filtering to separate the pericarp from the starch. One method and system embodiment uses a high-frequency, rotor-stator shearing device in the treatment of starch. This type of device produces high-shear, microcavitation forces which defibrillate the starch fed into it. Two commercially available high-frequency, rotor-stator dispersion devices are the Supraton™ devices manufactured by Krupp Industrietechnik GmbH and marketed by Dorr-Oliver Deutschland GmbH of Connecticut, and the Dispax™ devices manufactured and marketed by Ika-Works, Inc. of Cincinnati, Ohio. These devices are mentioned to provide examples of suitable devices but are not meant to limit acceptable high-frequency, rotor-stator shearing devices usable in the processes of the present invention.

To prepare the starch and any remaining pericarp for shearing, the starch is first reduced to a manageable size by grinding. Grinding to a desired particle size is accomplished in one or more stages. In a general aspect of the process, the milled starch/pericarp is ground by conventional hammer milling to a particle size sufficiently small enough to pass through a number 4 mesh sieve.

In one embodiment, the ground product is mixed with water to obtain a slurry of a preselected solids content. One of the purposes of this part of the process is to swell and further to defibrillate the starch. In one embodiment, the ground starch is fed into a hopper and is conveyed to a mixer-grinder-pump. Water is added to form a slurry having a solids content ranging from about 10% to about 25% solids. In one embodiment, the mixer-grinder-pump is a medium shear, rotor-stator device capable of mixing and pumping high solid content slurries. This device further reduces the particle size of the starch, wets the particles thoroughly with water, and disperses the particles within the water. Examples of this type of device are the HED™ manufactured and marketed by Ika Works, Inc. of Cincinnati, Ohio and the Gorator™ manufactured by Krupp Industrietechnik GmbH and marketed by Dorr-Oliver Deutschland GmbH of Connecticut.

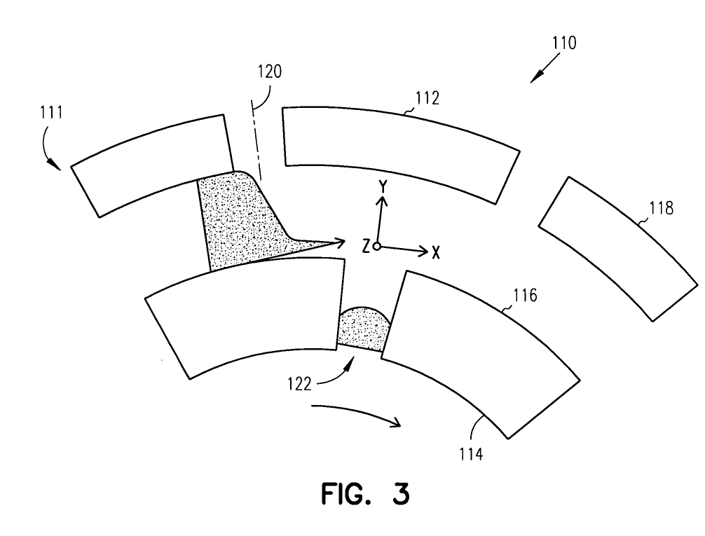

Referring to FIG. 3, a starch slurry is fed into the high-frequency, rotor-stator device 111 and is forced into a chamber 110. Inside the chamber is a series of coaxial meshing rings. The rings are configured with teeth, slots or bore holes. The rings configured with teeth are generally known as tooth and chamber tools and those configured with bore holes are generally known as nozzle tools. Generally, tooth and chamber tools are attached to both the rotor and the stator when tooth and chamber tools are used. When nozzle tools are used, generally, a tooth and chamber type tool is affixed to the rotor and a nozzle tool will be affixed to the stator.

The rings are concentric, radiating out from the center. The rings 112 on the stator are fixed and the rings 114 on the rotor are rotated by a shaft coupled to a motor.

The structure identified as 116 is representative of a tooth on a tooth and chamber tool attached to the rotor. The structure identified as 118 is representative of both a tooth on a tooth and chamber tool attached to the stator and the body of a nozzle tool spaced between bore holes. Accordingly, the space identified as 122 represents the gap between the teeth on a tooth and chamber tool attached to the rotor. The space identified as 120 represents both the gap between teeth on the stator tooth and chamber tool attached to the stator and the gap formed by a bore hole in a nozzle tool attached to the stator. The rings 114 on the rotor and the rings 112 on the stator are closely spaced at close tolerances. The space between the rotor and stator is typically about 1 mm.

Regarding a tooth and chamber tool, adjacent pairs of teeth are separated by gaps 120 and 122. The tooth and gap size determine the coarseness of the machine, i.e., a coarse tool has fewer teeth with larger gaps between adjacent teeth when compared with a medium or fine tool. Both the Supraton™ and Dispax™ allow the use of coarse, medium, and fine toothed rings in the same device, or the devices can have all coarse, all medium, or all fine toothed rings in the chamber so that the machines may be used in series, if desired. The use of multiple devices in series is one alternative to the use of a single device for processing starch.

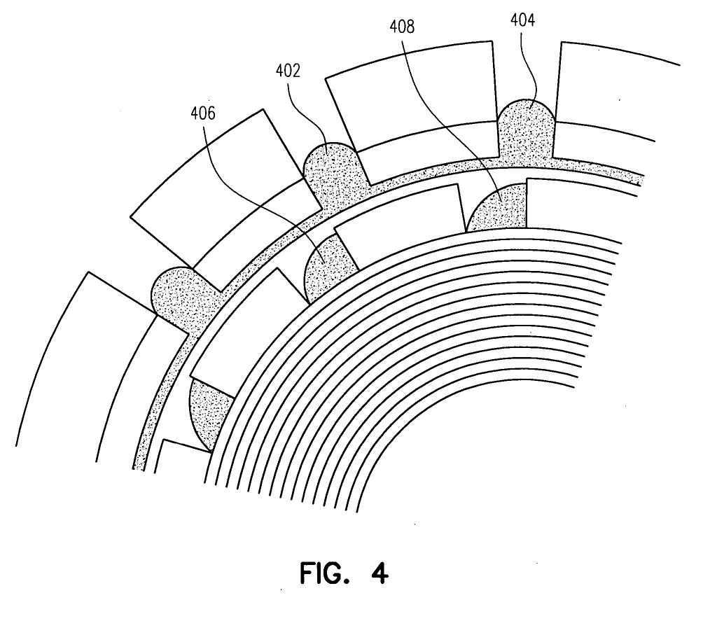

As the starch slurry is pumped under pressure into the chamber 10 by the mixer-grinder-pump, the slurry encounters each concentric layer of the tools in place in the chamber as the slurry is forced laterally. This lateral force is created by the pressure on the slurry as it is pumped into the chamber by the mixer-grinder-pump and by the centrifugal force created by the spinning rotor. The slurry passes through the gaps between the teeth as the rotor spins past the gaps in the stator. Flow is most pronounced when the gaps 122 between the rotor teeth align with the gaps 120 in the stator. The result is a pulsing flow with a rapid succession of compressive and decompressive forces. The starch material in the slurry is subjected to these repeated forces, as the centrifugal force accelerates it through the gaps toward the outer edge of the chamber. As the slurry moves towards the outer edge of chamber 110 the centrifugal forces increases, thus intensifying the forces generated in gaps 120 and 122. The repeated compressive and decompressive forces create microcavities in the slurry with extremely intensive energy zones. These zones are illustrated at 402, 404, 406 and 408 in FIG. 4. The starch structures are ripped apart by these forces. Additionally, the resulting starch structures exhibit extensive internal decrystallization due to the forces generated in the microcavities.

As the starch particles pass outward through the various gaps, the particles come in contact with the teeth and the body of the nozzle tool. Accordingly, some grinding of the particles may occur due to such contact. The grinding effects are relatively small, however, when compared with the combined effects of shear forces and microcavitation. Nonetheless, as solids loadings increase the instance of grinding may also increase.

Grinding typically cuts, slices, and dices fibrous material perpendicular to the fiber bundle, producing a more spherical type of particle. Shear forces in combination with microcavitation, on the other hand, tend to shatter the material, that is, they rip the fibers apart from the inside-out explosively forming irregularly shaped particles. Examination of these particles show them to have been “cut” both perpendicular to the fiber axis and longitudinally along the fiber axis. The effect on the starch particles is to shatter their structure, possibly disrupting the starch bonding without the compressive effects of grinding. Solids loadings not exceeding 30% are employed to minimize grinding of the starch and thus the compressive effects of the grinding.

While the precise mechanisms occurring within the chamber of the high-frequency, rotor-stator device are not totally understood several factors are thought to aid in explaining the effects on the treated starch. The swelling effect of liquids, particularly water, is thought to aid in creation of longitudinal shearing effects in the treated starch. The repeated compressive and decompressive events in and between the gaps are thought to create internal pressures tending to explode the starch particles and thus the starch structure thereof. It is also hypothesized that a harmonic resonance effect may be created during operation of the rotor-stator device in the sonic range. Thus, a harmonic frequency of a particular length when reached during processing would cause the effected particles to resonate and tend to aid in the destruction of the particle structure of the starch and any remaining pericarp.

As previously stated, high-frequency, rotor-stator dispersion devices may have differently configured rings or “tools” within the chamber. These tools, for example, may vary in the gap size between the teeth on the rings or in bore hole size in the case of a nozzle tool. With a larger gap size, the resulting material is more coarse than with a smaller gap size. As stated earlier, these tools can be varied within one device to contain coarse, medium, and fine rings in the chamber of the device. Likewise, a device may contain rings of the same rating so that the devices can be staged. This capability is important for use in a continuous process.

Once the starch is treated by a high-frequency, rotor-stator dispersion device, the treated starch is optionally subjected to pressurization and depressurization. The process of pressurization and depressurization with saturated steam forms hydrolysates (from hydrolysate fractions having the highest water content to hydrolysate fractions having the lowest water content. Once extracted, the fractions are cooled to ambient temperature. For some embodiments, the fractions are kept hot for further processing. With this method, there is a minimal loss in biofunctionality and bioresponse, as compared to traditional wet chemistry methods of separation. What is remarkable and unexpected is that this biofunctionality and bioresponse is achieved without complicated chemical treatment. Separation without loss of functionality and response is achieved by a one step steam pressurization/depressurization. What is also remarkable is that the extraction occurs with virtually any biomass feedstock. Pretreatment of biomass is minimal and is typically limited to size reduction.

The starch is then subjected to fermentation using conventional fermentation equipment and procedures.

For some embodiments, fractions obtained from fermentation are then subjected to ion exchange for the final purification.

It is believed that with this treatment, substantially native physical and chemical properties and structure are preserved for molecules such as native cellulose. It is also believed that with this treatment, a mass balance can be performed over a plant for virtually all of the bio-functional materials within the plant. Products extracted are in a concentration and having a reactivity within a range of what is predictable from a mass balance.

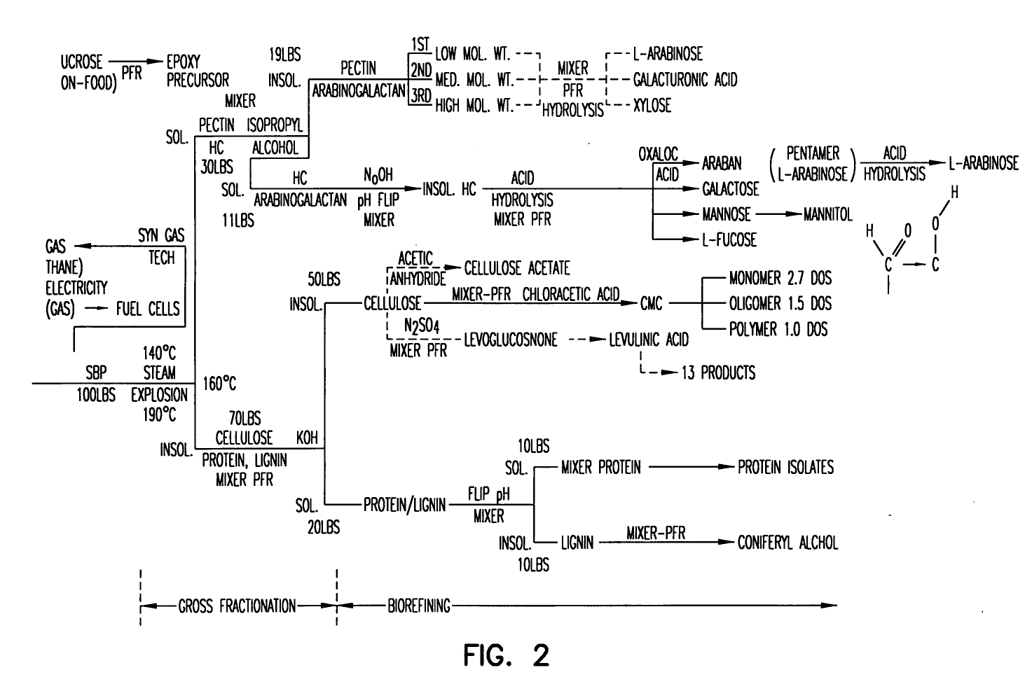

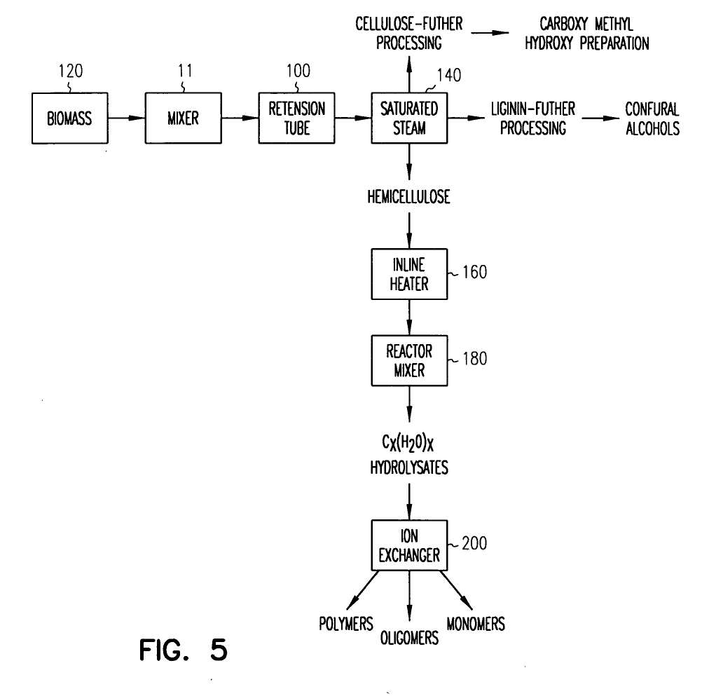

The present invention also includes a process for extracting, separating, and purifying individual stereoisomers and other specialty chemicals from components such as the germ or the pericarp. The process, illustrated schematically in FIG. 2 and at 100 in FIG. 5, comprises providing a source of biomass 120, such as corn germ after oil has been extracted and the pericarp, subjecting the biomass to saturated steam pressurization/depressurization 140 that increases surface area of the biomass and that permits separation of lignin, cellulose, and hemicellulose components from the biomass, heating hemicellulose 160 separated from the biomass in order to hydrolyze the hemicellulose and obtain hydrolyzed monomers, oligomers and polymers, and separating polymers, oligomers, and monomers from hydrolyzed hemicellulose 180. While hydrolysates are described, it is understood that the process of the present invention is usable to extract, separate and purify constituents and derivatives of cellulose, hemicellulose and lignin. For instance, cellulose derivatives such as carboxy methyl cellulose and hydroxypropyl cellulose can be obtained using the process of the present invention. Coniferyl alcohols are also obtainable. Stereoisomers of the monomers are further extracted using chromatographic methods 200.

The present invention achieves high yields of stereoisomers, when present, such as L-arabinose, using physical processes in addition to hydrolytic reactions, rather than exclusively conventional, water based, chemical extraction techniques. It has surprisingly been found that employing heat and pressure in treating biomass, such as corn germ and pericarp, increases production rates and percent yield of stereoisomers as compared to conventional, water based, chemical extraction processes.

As used herein, “simple sugars” refer to monosaccharides and oligosaccharides which are not decomposed into smaller sugars upon hydrolysis. Monosaccharides include pyranoses and furanoses. Monosaccharides are also classified according to the number of carbons in the molecule; for example, d,I-arabinose is a heptose.

As used herein, “complex sugars” refer to polysaccharides which are carbohydrates of high molecular weight capable of being hydrolyzed into a large number of monosaccharide units. Typical polysaccharides are cellulose, lignin, hemicellulose, starch and pentosan.

An oligosaccharide is a simple polysaccharide with a known number of constituent monosaccharide units, such as 1 to 10 monomers.

The term “biomass” as used herein refers to plant materials including, but not limited to corn germ and pericarp. Biomass in the form of plant materials includes cellulose and hemicellulose, both of which are polysaccharide, and lignin. Cellulose molecules are linear and unbranched glucose polymers with a high degree of polymerization between 10 and 10⁶. Cellulose has a strong propensity to form both intermolecular and intramolecular hydrogen bonds. Cellulose is stable against degradation under most physical and chemical conditions. Hemicellulose comprises heteropolysaccharides which are formed by a variety of different monomers. Most commonly the monomers are glucose, galactose, mannose, xylose and arabinose. Hemicellulose molecules have a degree of polymerization of about 10⁶. The term “feedstock” as used herein refers to any material supplied to a device, machine, or processing plant.

The biomass is subjected to a particle size of reduction to a size of chips or finer, such as a size of sawdust, using conventional particle reduction equipment. The smaller the size, the easier it is to mechanically handle the biomass. Smaller sized particles have a greater surface area and are more amenable to chemical reaction. Also, desired processing temperatures are reached more rapidly when using smaller particles.

In one embodiment, the biomass is fed into the high-frequency, rotor-stator device, illustrated schematically at 11 in FIG. 1 and is forced into a chamber 110, shown in FIG. 3. Inside the chamber is a series of coaxial meshing rings. The biomass treatment in the high-frequency, rotor-stator device is described above.

In one embodiment, the biomass is fed to a hopper following treatment in the rotor-stator device. The biomass may optionally be sprayed with water either before transfer to the hopper or while in the hopper. The biomass exits from the bottom of the hopper into a conveying feeder which contains a conveying mechanism such as a feed screw driven by a variable feed drive. The feed screw or other conveying mechanism feeds the material into a compacting feed tube and then into a pressurized retention tube, where the biomass particles are formed into a solid plug of material. The solid plug is compressed by surface pressures of up to 2000 psi.

The biomass is mechanically compacted prior to its introduction into the digester. The biomass is desirably in a moistened condition. The mechanical compaction removes air from the material prior to its introduction to steam pressurization. Air is undesirable because oxygen in the air tends to oxidatively degrade the biomass. Air also exerts a partial pressure and retards temperature and pressure equalization within the reactor.

Steam pressurization, within the pressurized reaction vessel, is typically operated with automatic pressure and temperature control systems. The partial pressure of any air pockets decreases steam pressure and temperature in the reactor below a preselected value. Compaction, followed by processing conditions discussed below, causes a degree of fibrillation of the biomass. Fibrillation of biomass assists in the heat transfer within and around the material.

Next, the biomass particles are disintegrated by steam pressure treatment and defibrination. In particular, the particles are treated with saturated steam at a temperature of from about 160 to 230 degrees Centigrade for a period of time from 2 minutes to 4 hours. The biomass is disintegrated by this steam treatment. In general, the lower the temperature used, the longer the duration of treatment should be. Thus, for some extractions, it is desirable to treat a biomass at 160 degrees Centigrade for about 4 hours. For other extractions, it is desirable to treat a biomass for 2 minutes at 230 degrees Centigrade.

This steam treatment separates fractions within biomass by most to least water content. The fractions are separated as extractables such as terpenes, fatty acids and so forth, lignin, pectin, hemicellulose and native cellulose. This steam treatment yields fractions at yields that are predictable by a mass balance of the biomass. In other words, the steam treatment and extraction of the present invention permits a user to ascertain bioactive/biofunctional materials present in living biomass and to extract the bioactive/biofunctional materials in quantities that approach or are substantially the same as the materials are present in the native biomass.

The rings are concentric, radiating out from the center. The rings 112 on the stator are fixed and the rings 114 on the rotor are rotated by a shaft coupled to a motor.

Biomass disintegrated this way is then, subsequently, for some embodiments, lixiviated with an aqueous solution of alkali. The concentration of NaOH is typically no greater than about 4% by weight.

The biomass mixture contains between 1 and 20 grams of water per gram of dry biomass and preferably about 16 grams of water per gram of dry biomass. In one embodiment the biomass mixture contains between 2 and about 50 grams of calcium hydroxide per 100 grams of dry biomass and preferably contains 30 grams of calcium hydroxide per 100 grams of dry biomass. In another embodiment the biomass mixture contains between 2 and 50 grams of alkali, hydroxide of sodium or hydroxide of potassium, per 100 grams of dry biomass.

The steam pressure treatment is performed in either a continuous stream or a batch type steam pressure reactor. In one embodiment, the reactor is manufactured by Stake Technology Ltd. Of Ottawa, Canada. One particular device is described in U.S. Pat. No. 4,136,207, which issued Jan. 23, 1979, and which is herein incorporated by reference. The steam pressure treatment is performed in the reactor vessel. The reactor vessel is maintained at a pressure that is between about 200 and 450 psig. The temperature in the reactor is maintained between about 390(F and 460(F. The biomass is fed intermittently for some embodiments and continuously for other embodiments. By varying the biomass stream but maintaining the reactor vessel conditions, the method of the present invention introduces an efficiency to the process, by avoiding ramp up and ramp down conditions within the reactor vessel.

The biomass is introduced into the reaction vessel in a manner that forms a solid plug at the inlet of the vessel. In one embodiment, the solid plug is formed in a device, such as a retention tube. The biomass plug prevents a loss of pressurization in the vessel. The combination of the biomass plug and constant pressurization permits instantaneous steam penetration of the biomass within the reaction vessel, and thus permits better control of processing times.

The biomass is processed at the steam temperatures described for a period of at least about 15 seconds and for some embodiments, at least about 5 minutes. The maximum time is about one hour.

After cooking, the biomass is cooled and depressurized substantially instantaneously. The biomass is in a moisture saturated condition. The biomass is subjected to sudden and substantially instantaneous decompression and adiabatic expansion, e.g. by discharging a small quantity of cooked biomass into ambient conditions.

The process of instantaneous pressurization and de-pressurization separates the biomass into components of lignin, cellulose and hemicellulose. The hemicellulose product is separated from the cellulose product and lignin product by techniques known in the art. It is further contemplated that the cellulose product is separated from the lignin product by techniques in the art.

Once the hemicellulose is extracted from the biomass, the hemicellulose for some embodiments, is heated in a steam heater, such as a Komax steam heater and then is hydrolyzed in a static mixer, such as a Komax reactor/static mixer, manufactured by Komax Systems, Inc., of Long Beach, Calif. One reactor/static mixer embodiment is described in U.S. Pat. No. 6,027,241, which is herein incorporated by reference. The reactor/static mixer is, in one embodiment, constructed so that an additive, such as sodium hydroxide is added countercurrent to the main fluid stream. The heater and mixer comprise a heater-mixer system.

Within the reactor, at approximately 329(F hemicellulose undergoes a phase transition, depending upon the moisture content, from a solid to a non-Newtonian fluid, somewhat like tooth paste. At temperatures higher than approximately 500° F., depending upon moisture content, the hemicellulose begins to pyrolize. Furthermore, the xylan component of the hemicellulose is degraded at temperatures above 428° F. Hence, to preserve the quality of the hemicellulose product stream, the hemicellulose exposure to temperatures above 356° F. should be as short as possible. The in-line reactor heater—static mixer system raises the temperature of the hemicellulose to between 329° F. and 347° F. The time to bring the temperature within this range is typically less than about 10 seconds to about 20 seconds.

Once heated, the hemicellulose is reacted with NaOH in the reactor/static mixer. The static mixer accepts the hemicellulose, a high viscosity stream and NaOH, the low viscosity stream. The NaOH is injected into the high viscosity stream, mixed by static mixing and a chemical reaction occurs between the alkali and the hemicellulose. In particular, the NaOH hydrolyzes the hemicellulose. The process of the present invention, unlike conventional sugar extraction processes, does not rely upon chemical reactions for extraction. Instead, the process of the present invention utilizes both sophisticated mechanical separation, occurring in the static mixer, coupled with NaOH addition for hydrolysis, for extraction and formation of hydrolysates.

In one embodiment, the hydrolysates include dissolved solids at a concentration of about four weight percent. The specific cation analysis included magnesium in a concentration of 2.5 ppm; calcium in a concentration of 3.66 ppm; potassium in a concentration of 0.40 ppm and sodium in a concentration of 1.81 ppm. The hydrolysate is prepared at 205 degrees Centigrade for three minutes with no acid.

The oligomers in the hydrolysates are converted to monomers without the addition of acid and are separated, in some embodiments, by ion exchange. In one embodiment, ion exchange columns are one inch in diameter and contain resin that is 3-4 feet deep. Ion exchange beds include a first cation resin bed and a second bed. Ion exchange beads are styrene crosslinked with divinyl benzene. Flow through the ion exchange bed is at 60 to 80 degrees Centigrade and is retained in the bed for a fifteen minute retention time. There is then a total retention time of thirty minutes through both of the beds. The conversion of the oligomers to monomers in the hydrolysate is then measured. If conversion not at a preselected value, the hydrolysate effluent is heated to 80 degrees Centigrade and samples are taken until conversion is completed. In one embodiment, the liquor provided to the ion exchange beds is a mixture of oligomers (⅗) and monomers (⅖).

If acidity is insufficient to complete the conversion, acid is added to the cation effluent to drive the reaction. The cation effluent pH is typically within a range of 2.5 to 3.5. Post hydrolysis is performed mildly with acid and once oligomers are converted to monomers, the reaction mixture is concentrated by vacuum evaporation to 50 percent dissolved solids.

With the process of the present invention, the hydrolysate mixture is further hydrolyzed to the basic monomeric unit from oligomers, and polymers in a single step and then separated on the basis of stereoisomer, i.e. optical or chirally pure monomer separation, in a second step. In another embodiment, the hydrolysate mixture is separated and a desired stereoisomer may be extracted in a single step.

In one embodiment of the process of the present invention, sugar products obtained by ion exchange are crystallized. In one embodiment, the crystallization is performed using a low intensity ultrasonic agitation. It is believed that this crystallization produces a product wherein crystals have few inclusions, are uniform in shape, in size, in density and in purity.

In one embodiment, the L-arabinose is separated from other monomers using the ion exchange methods and resin described herein. In another embodiment, the L-arabinose is separated from a mixture of hemicellulose hydrolysates.

For some embodiments, the high-frequency, rotor-stator treatment is used-in a process that includes the ion exchange embodiment. For other embodiments, the process includes one of either the high frequency, rotor-stator treatment or the ion exchange embodiment.

The present invention may be embodied in other specific forms without departing from its spirit or essential characteristics. The described embodiments are to be considered in all respects only as illustrative and not restrictive. The scope of the invention is, therefore, limited only by the appended claims rather than by the foregoing description. All changes which come within the meaning and range of functional equivalency of the claims are to be embraced within their scope.

1. A method for separating a germ fraction from a corn kernel, comprising: Milling the corn kernel to make a milled corn kernel; and Separating the germ fraction from the milled corn kernel by adding the kernel and germ fraction to an isotonic solution wherein the germ fraction rises to the top of the solution and the remaining milled corn kernel sinks to the bottom of the solution.

2. The method of claim 1 wherein the isotonic solution comprises fermentable sucrose and water.

3. The method of claim 1 wherein the isotonic solution is enclosed in a vessel.

4. The method of claim 3 wherein the germ is continuously removed from the top of the vessel.

5. The method of claim 3 wherein the remainder of the corn kernel is continuously removed from the bottom of the vessel.

6. The method of claim 1 wherein the isotonic solution comprises a recyclable material and water.

7. The method of claim 1 further comprising adding water to the corn kernel prior to milling.

8. The method of claim 1 wherein the milling is performed in a dry mill.

9. A corn germ separated from a corn kernel, the corn germ retaining substantially all of the oil held by the germ when it was part of the corn kernel.

10. A system, comprising: a dry mill; and a vessel and an isotonic solution contained in the vessel.

11. The system of claim 10, further comprising a water dispersing device.

12. The system of claim 10 wherein the isotonic solution is a sucrose solution.

13. The system of claim 10 further comprising a fermentation device.

14. The system of claim 13 wherein the isotonic solution is recyclable within the fermentation device.

15. The system of claim 10 further comprising a device for shearing particles.
